(19) United States
(12) Patent Application Publication
(10) Pub. No.: US 2024/0339213 A1
(43) Pub. Date: Oct. 10, 2024

(54) STAND-ALONE WEB BROWSER-BASED OR APP-BASED TREATMENT PLAN VIEWER

(71) Applicant: Hirsch Dynamics Holding AG, Wollerau (CH)

(72) Inventor: Markus HIRSCH, Wollerau (CH)

(73) Assignee: Hirsch Dynamics Holding AG, Wollerau (CH)

(21) Appl. No.: 18/749,694

(22) Filed: Jun. 21, 2024

Related U.S. Application Data
(63) Continuation of application No. PCT/EP2021/087540, filed on Dec. 23, 2021.

(51) Int. Cl. G16H 40/67; G16H 20/40

(52) U.S. Cl. CPC G16H 40/67; G16H 20/40

(57) ABSTRACT

An arrangement of orthodontic treatment planning software to create a treatment plan installed on at least one server and at least one local client is in the form of a plug-in running on a processing unit of a local computer or a mobile device. The at least one client has a user interface that visualizes a virtual representation of a three-dimensional virtual model of at least part of a dentition. The at least one server has a first data transmission interface for sending and receiving data via a data network and the local computer or mobile device has a second data transmission interface for sending and receiving data via a data network. The at least one client transmits a digital data record of a modified three-dimensional virtual model of the dentition and/or with information for a treatment plan via the second data transmission interface to the first data transmission interface.

### CROSS REFERENCE TO RELATED APPLICATIONS

This application is a continuation of and Applicant claims priority under 35 U.S.C. § 120 of International Application No. PCT/EP2021/087540 filed on Dec. 23, 2021, The international application under PCT article 21(2) was published in English. The disclosure of the aforesaid International Application is hereby incorporated by reference.

### BACKGROUND OF THE INVENTION

The present invention relates to an arrangement of at least one orthodontic treatment planning software to create a treatment plan installed on at least one server and at least one local client in the form of a plug-in running on a processing unit of a local computer or a mobile device, whereby the local computer or mobile device and the at least one server are in signal-conducting data connection or can be brought into a signal-conducting data connection. Furthermore, the present invention relates to a web browser-based or app-based method for visualizing at least one three-dimensional virtual model of at least part of a dentition by means of such an arrangement. Moreover, the present invention relates to a computer program product and a computer-readable medium.

A server comprising an orthodontic treatment planning software is shown by prior art document U.S. Pat. No. 8,260,591 B2, whereby a user—a lab staff or a doctor—can manipulate a three-dimensional virtual model in order to generate a treatment plan that is used by a doctor to treat a patient's orthodontic condition.

A disadvantage of the prior art is that this system is connected to a particular treatment planning software and hence uses an algorithm limited to this specific treatment planning software. Moreover, if a user wants to display the three-dimensional virtual model, the user needs to access the whole functionality of the system since the system comprises the treatment planning software to generate an adapted treatment plan by means of the three-dimensional virtual model. Furthermore, treatment planning software need a high amount of processing power as well as a high reliability to ensure a required quality of the treatment plan to be generated.

### SUMMARY OF THE INVENTION

It is an object of the present invention to provide an arrangement as well as a web browser-based or app-based method for visualizing at least one three-dimensional virtual model of at least part of a dentition that are appropriate to eliminate at least some of the disadvantages of the prior art, are particularly flexible, use resources such as processing power efficiently and/or are applicable to a broad field of application or rather various input files and/or ensure a high quality of desired treatment plans.

The object of the present invention is accomplished by a method and arrangement of at least one orthodontic treatment planning software (1) to create a treatment plan (15) installed on at least one server (2) and at least one local client (3) in the form of a plug-in running on a processing unit (30) of a local computer (4) or a mobile device (5), whereby the local computer (4) or mobile device (5) and the at least one server (2) are in signal-conducting data connection (6) or can be brought into a signal-conducting data connection (6), wherein:
the at least one client comprises a user interface in the form of a stand-alone web browser-based or app-based treatment plan viewer configured to visualize at least one virtual representation of at least one three-dimensional virtual model of at least part of a dentition
the at least one server and the local computer or mobile device are spatially separated from each other, whereby the at least one server comprises a first data transmission interface for sending and receiving data via a data network and the local computer or mobile device comprises a second data transmission interface for sending and receiving data via a data network, whereby the first data transmission interface and the second data transmission interface are separated from each other
the at least one client is configured to transmit at least one digital data record of a modified three-dimensional virtual model of the at least part of the dentition and/or with information for a desired treatment plan of the at least part of the dentition by means of the second data transmission interface of the local computer or mobile device to the first data transmission interface of the at least one server.

By means of the transmission of the at least one digital data record, the arrangement enables a doctor to communicate a desired treatment plan or approve a specific treatment plan, whereby the doctor is able to display the required information like the treatment plan or the three-dimensional virtual model resource-saving on devices with low processing power without affecting the quality of the treatment plan (which is essential for required quality standards especially in the field of orthodontics of example). Moreover, the doctor can consider the proposed treatment plan visually in a user-friendly way remotely from the at least one server that comprises the treatment planning software, whereby the number of servers is in general arbitrary and the local client can communicate with all servers or rather all treatment plans of these servers by means of the second data transmission interface. It is also possible that a doctor guides a trained staff to generate a desired treatment plan within the at least one server.

The information of the at least one digital data record is in general arbitrary and can comprise comments, desired amendments of the treatment plan or rather particular tooth movement steps of the treatment plan or modified three-dimensional virtual models for the use within the at least one treatment planning software. An information for a lab—which supervises the treatment plans—that the proposed treatment plan is a proved by the doctor can be sufficient as well. By the at least one digital data record, a guarantee can be ensured that the treatment plan that finally is used for a treatment of a patient's dentition is at least double-checked.

The lab can adapt the treatment plan according to a doctor's wishes by means of the treatment planning software of the at least one server without requiring the doctor to install or enter a treatment planning software at all by simultaneously meet predefined quality benchmarks which are required to treat a patient appropriate and/or save resources at the doctor's end.

More the at least one server can use a higher amount of computational power and less-complex and/or expensive devices for the local client can be used to visualize all relevant information for the doctor, whereby the doctor can nevertheless compare and change the at least one three-dimensional virtual model inside the viewer by means of a plug-in of the local client. After checking and/or manipulating the information provided by the at least one server, a final sign off and/or modification of the treatment plan through the lab by creating the corresponding treatment plan or approving the treatment plan is possible.

The arrangement ensures an open-ended solution which is independent of the specific treatment planning software and in particular of input digital data records and/or digital data records that are uploaded or exported by the local client. Hence, the stand-alone web browser-based or app-based treatment plan viewer of the local client can be provided to and interact with a wide variety of different servers and/or treatment planning software, which constitutes a particular flexible, user-friendly and simple to adjust system or rather arrangement.

Due to the fact, that a plurality of different treatment planning software and servers for processing the data records or rather file formats exist with respect to treatment plans, the procedure to interpret the individual file formats as well as the communication between the specific systems is cumbersome and error-prone. Moreover, an applicability of viewers connected to treatment planning software are limited to a finite number of systems or rather the related connections points between the systems. Furthermore, a conversion of the viewer is complex as well as problematic with respect to an appropriate processing of the sensible data to treat a patient and is difficult to adapt. However, the stand-alone web browser-based or app-based treatment plan viewer is configured to work as an interpreter between the local computer or mobile device and the various systems with their own different treatment planning software such that various input digital data records can be processed and digital data records can be exported to be used with these different systems.

The processing unit can be designed for example in the form of a processor comprised by a mobile device like a smartphone, a table, a notebook, a computer or like that for processing input digital data records and/or for visualization the at least one three-dimensional virtual model of the at least part of the dentition and/or the at least one input digital data record comprising the treatment plan by means of a monitor for instance. The processing unit can further cause the local computer or the mobile device to export the at least one digital data record.

Stand-alone web browser-based or app-based treatment plan viewer means in this context that the viewer does not comprise a treatment planning software on it's own and is in general not limited to a specific treatment planning software of the at least one server. Thus, STL files or OBJ files of a variety of different treatment planning software can be uploaded as an input for the viewer to export the at least one digital data record for the at least one server, whereby occlusal states can be visualized and adapted virtually to a desired state for a treatment plan to adjust a given orthodontic condition within the local client.

The stand-alone web browser-based or app-based treatment plan viewer can be provided for example in the form of an app or within a web-browser which allows a user to display a photorealistic tooth model of the three-dimensional virtual model of various movement steps of a treatment plan for the at least part of the dentition, whereby movement steps of the treatment plan can be visualized in the form of a tooth/teeth movement table for instance.

The plug-in can be implemented for example by use of protocols like FTP, SMTP and/or HTTP (for checking, transferring and/or translating digital data records), whereby it is particularly preferred provided that the plug-in acts as an interface between data transmission protocols of the local computer or mobile device and the at least one server using for example TCP/IP and/or UDP. It is particularly provided that the plug-in itself comprises at least one data transmission protocol for communication with data transmission protocols of the local computer or mobile device and the at least one server.

As stated above, protection is also sought after a web browser-based or app-based method for visualizing at least one three-dimensional virtual model of at least part of a dentition by means of such an arrangement, whereby the following steps are performed:
the local computer or mobile device receives at least one input digital data record by means of the second data transmission interface via a data network from the first data transmission interface of the at least one server
the at least one local client visualizes at least one virtual representation of at least one three-dimensional model of the at least part of the dentition by means of the user interface in the form of a stand-alone web browser-based or app-based treatment plan viewer
the at least one local client transmits at least one digital data record of a modified three-dimensional virtual model of the at least part of the dentition and/or with information for a desired treatment plan of the at least part of the dentition by means of the first data transmission interface to the second data transmission interface of the at least one server.

As stated above, protection is also sought after a computer program product, which, when the program is executed by a processing unit, causes the processing unit to carry out such a method.

As stated above, protection is also sought after a, particular non-volatile, computer-readable medium comprising instructions which, when executed by a processing unit, cause the processing unit to carry out such a method.

Preferred embodiments of the present invention are defined in the dependent claims. Features of the arrangement claims are applicable with respect to the web browser-based or app-based method claims and vice versa.

In a preferred embodiment of the present invention, it is provided that the at least one client is
free of any orthodontic treatment planning software and/or
configured to transmit the at least one digital data record comprising information suitable to amend the treatment plan to the at least one server, whereby it is particularly preferred that the at least one digital data record is based on a modification of the at least one three-dimensional virtual model of the at least part of the dentition within the at least one client and/or comprises information for a desired treatment plan for the at least part of the dentition.

In a preferred embodiment of the present invention, it is provided that the at least one client is configured to facilitate a user of the at least one client to approve the treatment plan or the at least one three-dimensional virtual model and/or to generate a desired treatment plan by adding notes to the treatment plan and/or
add at least one attachment to the at least one three-dimensional virtual model and/or
move objects, preferable teeth, of the at least one three-dimensional virtual model and/or
generate interproximal reduction on the at least one three-dimensional virtual model and/or
adjust the at least one three-dimensional virtual model and/or
move a plurality of objects of the at least one three-dimensional virtual model simultaneously and/or
add cutouts or slits to the at least one three-dimensional virtual model.

It is particularly preferred provided that the at least one digital data record comprises supplementary information of the at least part of the dentition, a virtual representation of a desired orthodontic condition and/or a desired treatment plan, whereby it is particularly preferred that the at least one digital data record is in the form of an OBJ file, an STL file, a CAD file and/or a CSV file.

Supplementary information can comprise information about the at least part of the dentition of interest like temporal steps of a proposed movement, patient's information and/or structural data with respect to the at least part of the dentition like segmented entities as teeth, gingiva or attachments—in particular together with their location to one another—for instance which can be identified by the local client. The local client can generate desired modification which can be notes for or within the treatment plan, comments, amended virtual representations for a desired change with respect to an improved treatment of the patient, screenshots of three-dimensional virtual models et cetera.

It is particularly preferred provided that the at least one client is configured to upload at least one input digital data record, particularly preferred an OBJ file, an STL file, a CAD file and/or a CSV file, for visualizing the at least one visual representation of the at least one three-dimensional virtual model and/or at least one treatment plan of the at least part of the dentition, whereby it is particularly preferred that the at least one client is configured to simultaneously upload a plurality of input digital data records, whereby each input digital data record comprises information about a movement step of a treatment plan of the at least part of the dentition.

In a preferred embodiment of the present invention, it is provided that the at least one digital data record and/or the at least one input digital data record comprises supplementary information of the at least part of the dentition, preferably in the form of an intraoral scan, a scan, an x-ray, a photograph, patient data, user comments, a number of trays, comments on interproximal reductions, a number or a location of attachments and/or a number of steps of the treatment plan.

In a preferred embodiment of the present invention, it is provided that the at least one client is configured to
identify at least one tooth based on the at least one input digital data record and/or the plurality of input digital data records and/or
translate the at least one input digital data record and/or the plurality of input digital data records to the at least one virtual representation, preferably of movement steps of the treatment plan, and/or
match at least one tooth with an area of the at least one three-dimensional virtual model, preferably of movement steps of the treatment plan, based on the at least one input digital data record and/or the plurality of input digital data records.

It is particularly preferred provided that the at least one client is configured to move and/or rotate the at least one virtual representation and to modify the at least one three-dimensional virtual model, particularly preferred by moving at least one tooth relative to at least one other tooth and/or gingiva and/or by locating at least one attachment to at least one tooth.

It is particularly preferred provided that the at least one virtual representation includes at least one attachment based on a treatment plan, measurements of at least one tooth and/or movement steps based on a treatment plan.

In a preferred embodiment of the arrangement and the method, it is provided that the at least one client comprises
means for importing, particularly preferred user interface, which are configured to import via a data exchange system and/or a case management system
at least one input digital data record of the at least one three-dimensional virtual model of the at least part of the dentition to be treated and/or
at least one input digital data record of a treatment plan of an orthodontic treatment planning software based on the at least part of the dentition and/or
at least one input digital data record of at least one three-dimensional virtual model of at least part of the dentition based on at least one digital data record of a treatment plan and/or movement steps of an orthodontic treatment planning software for the at least part of the dentition, and/or
means for processing at least one input digital data record, particularly preferred processing unit and/or interpreter between the local computer or mobile device and the at least one server, which are configured to
analyze the at least one input digital data record based on the file format and/or
identify at least one object of the at least part of the dentition and/or at least one attachment for the at least part of the dentition based on the at least one input digital data record and/or
match the at least one object and if applicable the at least one attachment to construct the at least one three-dimensional virtual model of the at least part of the dentition and/or
means for visualizing and/or modifying, particularly preferred web browser or app interface, which are configured
to visualize within a web browser or an app the at least one input digital data record, particularly preferred the treatment plan, movement steps of the treatment plan and/or the at least one virtual representation of the at least one three-dimensional virtual model of the at least part of the dentition to be treated, and/or
move and/or rotate the at least one virtual representation to a desired size and/or viewing angle and/or
modify the at least one three-dimensional virtual model of the at least part of the dentition and/or
means for exporting, particularly preferred user interface, which are configured to export
the at least one digital data record suitable for approving the treatment plan or creating a new treatment plan by means of the at least one server, whereby it is particularly preferred that the at least one digital data record is in the form of a modified input digital data record based on a modification of the at least one three-dimensional virtual model and/or a desired modification of at least one movement step of the treatment plan for the at least part of the dentition and/or
a virtual representation of at least one modified three-dimensional virtual model and/or
the treatment plan, preferably with notes for a desired treatment plan and/or information of discrepancies and/or measurements of objects of the at least one three-dimensional virtual model.

A treatment plan is a plan which comprises consecutive steps to move an orthodontic condition into a subsequent desired orthodontic condition—for example in the form of a table that is used for a treatment with an orthodontic appliance like an orthodontic aligner. To ensure that the viewer is independent from a particular treatment planning software, an improved process of uploading the STL or OBJ files that are usually inputted in the treatment planning software in a specific form adapted to the individual treatment planning software can be used.

In general, for each tooth movement step of the treatment plan one STL file or OBJ file has to be inputted iteratively for the treatment planning software and processed by the at least one server, whereby by means of the viewer it is possible to upload multiple STL files and/or OBJ files—generated by at least one treatment planning software—simultaneously to be visualized within the local client.

The local client is particularly preferred configured to analyze the at least one input digital data record by means of transformation matrix searching, preferably by outputting at least one digital data record comprising an updated transformation matrix.

OBJ and STL files have their own characteristics and can differ dependent on the treatment planning software by which they are generated. OBJ files can incorporate information like the color of structures but does not distinguish between individual structures like specific teeth and/or gingiva. STL files can provide a high-resolution representation of the geometry of a dentition but are in their mere form not suitable to represent a segmented orthodontic condition temporal, whereby the data structure that map to the dentition can differ significantly based on the used format. It is crucial to read these digital data records correctly and in the correct order to be able to visualize a virtual model of at least part of the dentition correctly with respect to the at least one digital data record and to prevent mistakes during the use of the arrangement which can have serious effects on the treatment of a patient's orthodontic condition.

A preferred preparation of the at least one digital data record can be exemplified as followed: A plurality of input digital data records for every tooth movement step of the treatment plan is uploaded by the local client. The input digital data records can already comprise segmented objects (OBJ files for instance) of the dentition of represent a surface-structure of the dentition (STL files or CAD files for instance). The input digital data records can comprise a movement table for each movement step of the teeth (CSV files for instance) and/or interproximal reduction values. Data of the following form have to be processed by the local client to identify every object of an arbitrary input digital data record to be matched with respect to the three-dimensional virtual model, whereby the explicit form can vary for a specific treatment plan software or rather the corresponding file format and interpreted by the local client accordingly:
f 35830 36817 36819
f 35781 35760 36820
#Object16: Tooth 37
g Tooth 37
o Tooth 37
x 18788 faces
f 37104 37105 37028
f 37131 37109 37170

It can be useful to provide a folder and/or sub-folders with a certain case ID in order to label the input digital data records and/or the digital data records for improved processing by means of the local client, whereby the input digital data records and/or the digital data records are labelled such that the viewer can process them in order to visualize the doctor the virtual representations for instance. Summary-files can be generated as an intermediate processing step.

Means for importing and/or means for exporting can be constituted for example by the second data transmission interface and/or a user interface of the viewer.

Means for visualizing and/or modifying can be constituted for example by a monitor of the local computer or the mobile device and/or can comprise a web browser interface.

Means for processing can be constituted for example by a processor or rather a processing unit, whereby it is particularly preferred provided that the the means for processing are configured to generate the at least one digital data record out of the at least one input digital data record, preferably by transforming the at least one input digital data record. used to generate the at least one digital data record. It is conceivable that the means for processing comprises a module for visualizing the at least one input digital data record and/or communicates with the means for visualizing and/or modifying.

In a preferred embodiment of the present invention, it is provided that
the means for importing are at least temporally in signal-conducting data connection with a DDX database and/or a CRM software, preferably by means of an API of the means for importing, and/or
the means for visualization are configured to display discrepancies and/or measurements of objects of the at least part of the dentition based on the treatment plan and/or on at least one modified three-dimensional virtual model DDX is a data exchange system—usually at a lab's end—and CRM is a case management system—usually at a doctor's end—whereby the local client is configured to access the relevant information with respect to the treatment plan and/or patient's individual data by use of an APL (application programming interface).

### BRIEF DESCRIPTION OF THE DRAWINGS

Features of the arrangement are applicable with respect to the method claims and vice versa. Further details and advantages of the present invention are explained in more detail below with reference to the description of the figures and with reference to the exemplary embodiments illustrated in the drawings. In the drawings, the figures show:

FIG. 1 shows an arrangement pursuant to a preferred embodiment in a schematic illustration with a server, a mobile device and a local computer,

FIGS. 2A, 2B, 2C, 3A, 3B, 4A, and 4B show a stand-alone web browser-based or app-based treatment plan viewer to be used in an arrangement according to FIG. 1,

FIG. 5 shows a schematic illustration of the local computer of the arrangement according to FIG. 1, and

FIG. 6 shows a digital data record exported by the local computer according to FIG. 1.

### DETAILED DESCRIPTION OF THE EMBODIMENTS

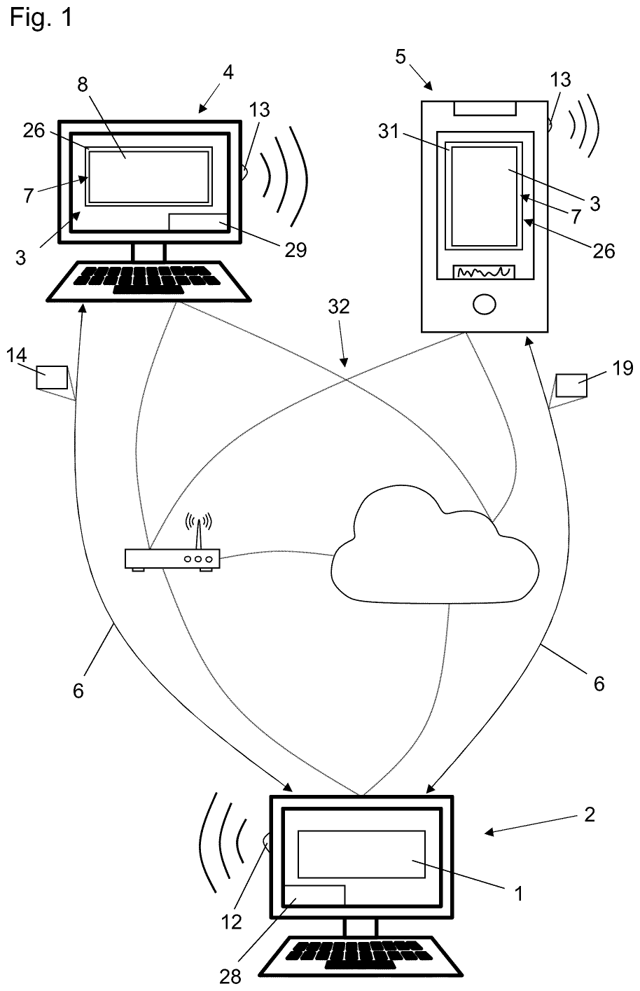

FIG. 1 discloses an arrangement of an orthodontic treatment planning software 1 to create a treatment plan 15 installed on a server 2 and two local clients 3 in the form of a plug-in running on a processing unit 30 of a local computer 4 and a mobile device 5. The local computer 4, the mobile device 5 and server 2 are in signal-conducting data connection 6, whereby the clients 3 comprise a user interface 7 in the form of a stand-alone web browser-based or app-based treatment plan viewer 8 configured to visualize virtual representations 9 of three-dimensional virtual models 10 of dentitions 11 or parts of the dentitions 11.

The server 2, the local computer 4 and the mobile device 5 are spatially separated from each other, whereby the server 2 comprises a first data transmission interface 12 for sending and receiving data via a data network 32. The local computer 4 and the mobile device 5 comprise a second data transmission interface 13 for sending and receiving data via the data network 32, whereby the first data transmission interface 12 and the second data transmission interface 13 are separated from each other.

The clients 3 are configured to transmit digital data records 14 of a modified three-dimensional virtual model 10 of the dentition 11 or with information for a desired treatment plan 15 of dentition 11 by means of the second data transmission interface 13 of the local computer 4 or mobile device 5 to the first data transmission interface 12 of the server 2. The client 3 is free of any orthodontic treatment planning software 1 and configured to transmit the digital data records 14 comprising information suitable to amend the treatment plan 15 to server 2. The digital data records 14 are based on a modification of the three-dimensional virtual model 10 of the dentition 11 within the clients 3 or comprise information for a desired treatment plan 15 for the dentition 11.

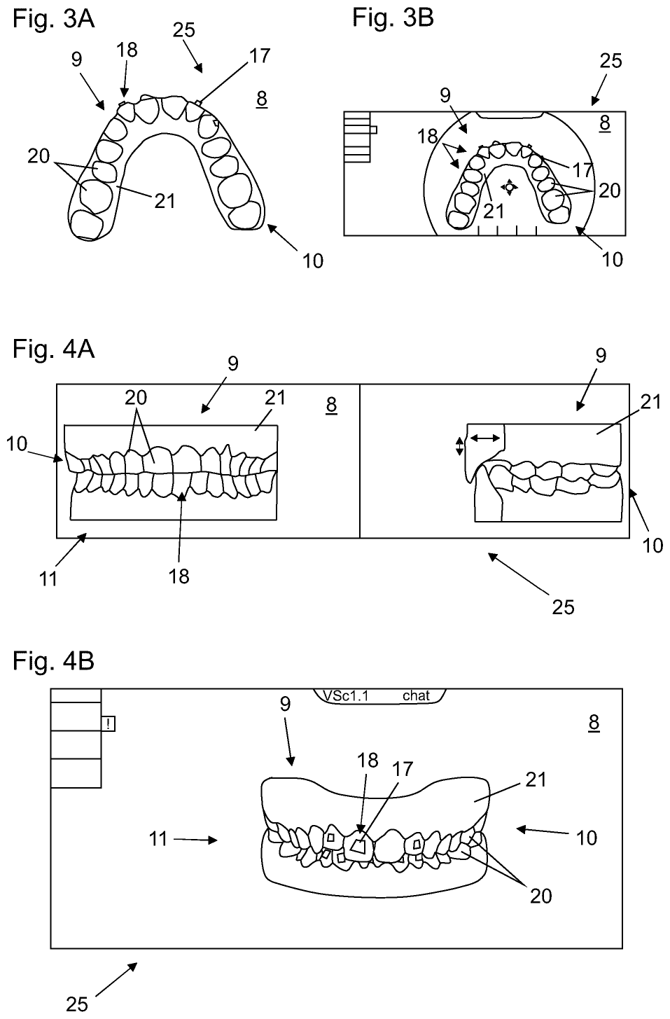

The local computer 4 and the mobile device 5 comprise means for importing 22 (cf. FIG. 5) to upload input digital data records 19 from the server 2 to the clients 3, whereby the means for importing 22 are at least temporally in signal-conducting data connection with a DDX database 28 and a CRM software 29 by means of an API of the means for importing 22.

The number of servers 3 is in general arbitrary, whereby the client 3 is capable of uploading input digital data records 19 from different treatment planning software 1 simultaneously or consecutively.

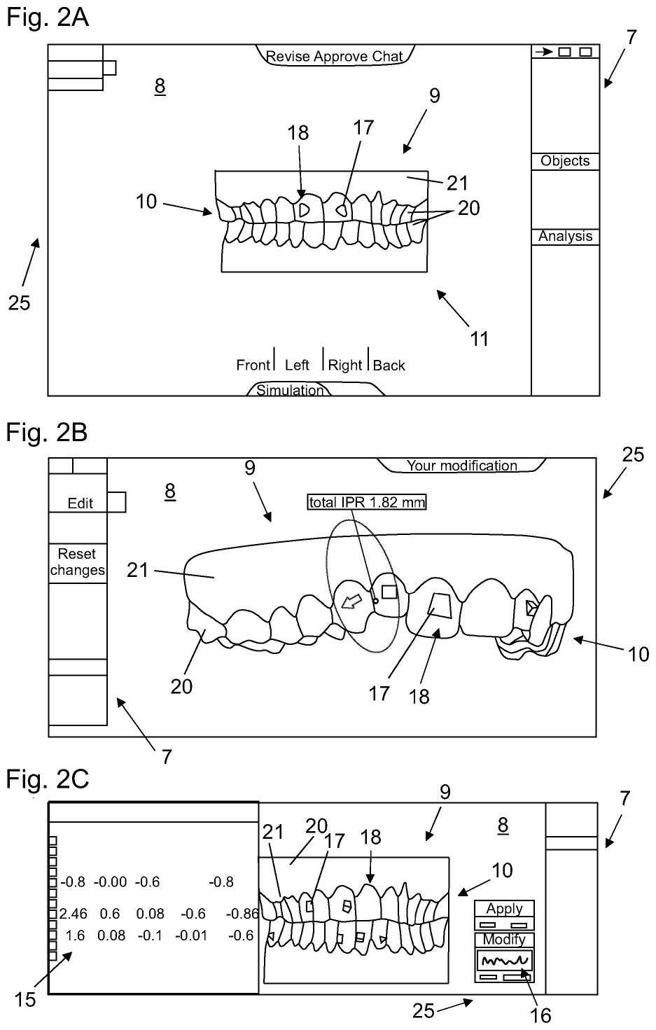

FIG. 2A discloses the stand-alone web browser-based or app-based treatment plan viewer 8 of one of the clients 3 which is configured to import input digital data records 19 of the three-dimensional virtual model 10 of the dentition 11 to be treated by means of a user interface 7 via a data exchange system or a case management system.

Input digital data record 19 of the treatment plan 15 of the orthodontic treatment planning software 1 based on the dentition 11 can be uploaded and input digital data records 19 of three-dimensional virtual models 10 of the dentition 11 based on digital data records of the treatment plan 15 or movement steps of the orthodontic treatment planning software 1 for the dentition 11 can be uploaded.

The local client 3 processes the input digital data records 19 by means of a processing unit 30 or an interpreter between the local computer 4 or mobile device 5 and the server 3 in order to
analyze the input digital data records 19 based on the file format,
identify objects 18 like teeth 20, gingiva 21 or attachments 17 of the dentition 11 based on the input digital data records 19 and
match the objects 18 to construct the three-dimensional virtual model 10 of the dentition 11.

The local client 3 visualizes by means of a browser or app interface 25 within a web browser 26 or an app 31 the input digital data records 19, the treatment plan 15, movement steps of the treatment plan 15 and virtual representations 9 of the three-dimensional virtual models 10 of the dentition 11 to be treated. A user of the local client 3 can move or rotate the virtual representations 9 to a desired size and viewing angle modify the three-dimensional virtual model 10 of the dentition 11.

The local client 3 exports by means of a user interface 7 the digital data record 14 suitable for approving the treatment plan 15 or creating a new treatment plan 15 by means of server 2. The digital data record 14 is in the form of modified input digital data records 19 based on a modification of the three-dimensional virtual model 10 or a desired modification of movement steps of the treatment plan 15 for the dentition 11.

FIG. 2B and FIG. 2C disclose that the virtual representation 9 includes attachments 17 based on a treatment plan 15, measurements of teeth 20 and movement steps based on a treatment plan 15. A user of the viewer 8 can add new attachments 17 or modify the virtual representation 9 for instance.

The client 3 is configured to facilitate a user of the client 3 to approve the treatment plan 15 or the three-dimensional virtual model 10 and to generate a desired treatment plan 15 by adding notes 16 to the treatment plan 15. However, the treatment plan 15 itself is not generated within the client 3 as no treatment planning software 1 is comprised by the stand-alone web browser-based or app-based treatment plan viewer 8.

For example, the user of the client 3 can
add attachment 17 to the three-dimensional virtual model 10
move objects 18 like teeth 20 of the three-dimensional virtual model 10
generate interproximal reduction on the three-dimensional virtual model 10
adjust the three-dimensional virtual model 10
move a plurality of objects 18 of the three-dimensional virtual model 10 simultaneously
add cutouts or slits to the three-dimensional virtual model 10.

FIG. 3A discloses functionalities with respect to the virtual representation 9, whereby the client 3 is configured to
identify teeth 20 based on the input digital data record 19 or the plurality of input digital data records 19
translate the input digital data record 19 or the plurality of input digital data records 19 to the virtual representation 9 or virtual representations 9 of movement steps of the treatment plan 15
match at least one tooth 20 with an area of the three-dimensional virtual model 10 or movement steps of the treatment plan 15 based on input digital data record 19 or the plurality of input digital data records 19.

The client 3 is configured to move and rotate the virtual representation 9 and to modify the three-dimensional virtual model 10 by moving teeth 20 relative to other teeth 20 or the gingiva 21 and by locating attachments 17 to teeth 20.

FIG. 3B differs with respect to FIG. 3A in that the user addresses another functionality, whereby the arch of the three-dimensional virtual model 10 can be adjusted.

FIG. 4A discloses that the client 3 comprises further functionalities, whereby the means for visualization 24 are configured to display discrepancies and measurements of objects 18 of the dentition 11 based on the treatment plan 15 or on a modified three-dimensional virtual model 10 for instance.

FIG. 4B discloses the web browser-based or app-based viewer 8, whereby the method for visualizing three-dimensional virtual models 10 of the dentition 11 or parts of the dentition 11 can be done by means of the arrangement for example as follows:
the local computer 4 or mobile device 5 receives input digital data records 14 by means of the second data transmission interface 13 via the data network 32 from the first data transmission interface 12 of server 2,
the local client 3 visualizes the virtual representation 9 of the three-dimensional model 10 of the dentition 11 by means of the user interface 7 in the form of the stand-alone web browser-based or app-based treatment plan viewer 8 and
the local client 3 transmits a digital data record 14 of a modified three-dimensional virtual model 10 of the dentition 11 or part of the dentition 11 or with information for a desired treatment plan 15 of the dentition 11 by means of the first data transmission interface 12 to the second data transmission interface 13 of the server 2.

FIG. 5 discloses that the client 3 comprises
means for importing 22 in the form of a user interface 7 and a computing unit of the local computer 4,
means for processing 23 input digital data records 19 in the form of a processing unit 30 of the local computer and an interpreter between the local computer 4 the server 2,
means for visualizing 24 and/or modifying in the form of a web browser 26 (or in the case of an standalone app-based viewer 8 an app interface 25) which is connected to a part of the processing unit 30 and
means for exporting 27 in the form of a user interface 7 connected to a computing unit of the local computer 4

The arrangement can comprise a computer program product, which, when the program is executed by the processing unit 30, causes the processing unit 30 to carry out the method for visualizing three-dimensional virtual models 10. The arrangement can comprise a volatile or non-volatile computer-readable medium comprising instructions which, when executed by the processing unit 30, cause the processing unit 30 to carry out the method for visualizing three-dimensional virtual models 10.

The client 3 is configured to upload input digital data records 19 like OBJ files, STL files, CAD files, CSV files et cetera for visualizing the visual representations 9 of the three-dimensional virtual models 10 or treatment plans 15 of the dentition 11. The client 3 is configured to simultaneously upload a plurality of input digital data records 14, whereby each input digital data record 14 comprises information about a movement step of a treatment plan 15 of the dentition 11.

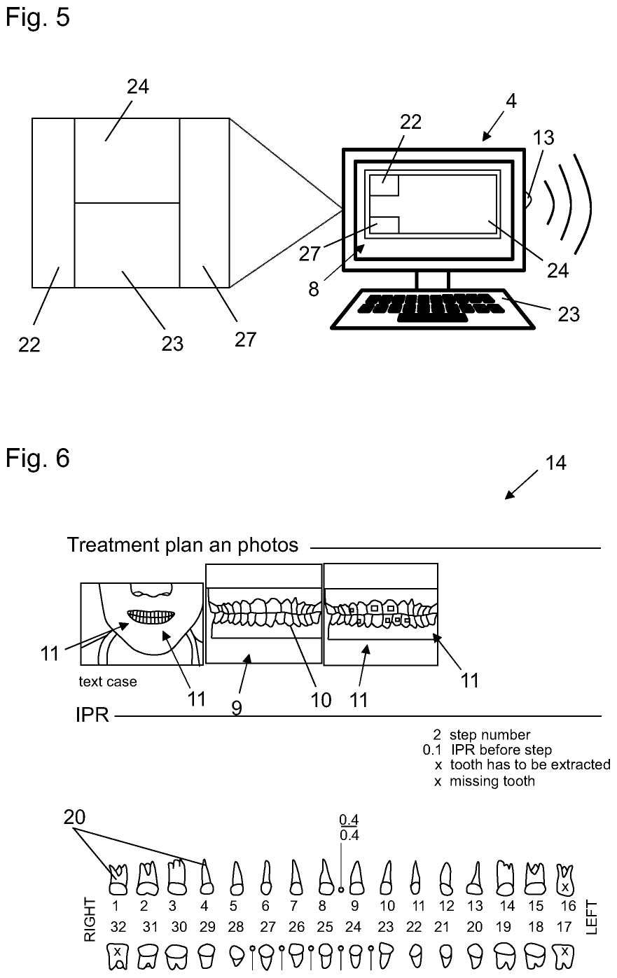

FIG. 6 discloses that—apart from digital data records 14 that are suitable to create a modified treatment plan 15 or approve the treatment plan 15—the digital data records 14 can comprise supplementary information of the dentition 11, a virtual representation 9 of a desired orthodontic condition and a desired treatment plan 15. The digital data record 14 can be in general in the form of an OBJ file, an STL file, a CAD file, a CSV file or like that.

The digital data records 14 and the input digital data records 19 comprise supplementary information of dentition 11 in the form of an intraoral scan, a scan, an x-ray, a photograph, patient data, user comments, a number of trays, comments on interproximal reductions, a number or a location of attachments 17 or a number of steps of the treatment plan 15.

The local client 3 has exported a virtual representation 9 of a modified three-dimensional virtual model 10 and can export the treatment plan 15 with notes 16 for a desired treatment plan 15 or information of discrepancies and measurements of objects 18 of the three-dimensional virtual model 10. This can be done in textual form, by means of a table or via a graphical visualization.

### LIST OF REFERENCE SIGNS

orthodontic treatment planning software (1)
server (2)
local client (3)
local computer (4)
mobile device (5)
signal-conducting data connection (6)
user interface (7)
stand-alone web browser-based treatment plan viewer (8)
virtual representation (9)
three-dimensional virtual model (10)
dentition (11)
first data transmission interface (12)
second data transmission interface (13)
digital data record (14)
treatment plan (15)
notes (16)
attachment (17)
object (18)
input digital data record (19)
tooth (20)
gingiva (21)
means for importing (22)
means for processing (23)
means for visualizing (24)
browser or app interface (25)
web browser (26)
means for exporting (27)
DDX database (28)
CRM software (29)
processing unit (30)
app (31)
data network (32)

What is claimed is:

1. An arrangement of at least one orthodontic treatment planning software (1) to create a treatment plan (15) installed on at least one server (2) and at least one local client (3) in the form of a plug-in running on a processing unit (30) of a local computer (4) or a mobile device (5), whereby the local computer (4) or mobile device (5) and the at least one server (2) are in signal-conducting data connection (6) or can be brought into a signal-conducting data connection (6), wherein:
the at least one client (3) comprises a user interface (7) in the form of a stand-alone web browser-based or app-based treatment plan viewer (8) configured to visualize at least one virtual representation (9) of at least one three-dimensional virtual model (10) of at least part of a dentition (11)
the at least one server (2) and the local computer (4) or mobile device (5) are spatially separated from each other, whereby the at least one server (2) comprises a first data transmission interface (12) for sending and receiving data via a data network (32) and the local computer (4) or mobile device (5) comprises a second data transmission interface (13) for sending and receiving data via a data network (32), whereby the first data transmission interface (12) and the second data transmission interface (13) are separated from each other, and
the at least one client (3) is configured to transmit at least one digital data record (14) of a modified three-dimensional virtual model (10) of the at least part of the dentition (11) and/or with information for a desired treatment plan (15) of the at least part of the dentition (11) by means of the second data transmission interface (13) of the local computer (4) or mobile device (5) to the first data transmission interface (12) of the at least one server (2).

2. The arrangement according to claim 1, whereby the at least one client (3) is free of any orthodontic treatment planning software (1) and/or
configured to transmit the at least one digital data record (14) comprising information suitable to amend the treatment plan (15) to the at least one server (2), whereby it is particularly preferred that the at least one digital data record (14) is based on a modification of the at least one three-dimensional virtual model (10) of the at least part of the dentition (11) within the at least one client (3) and/or comprises information for a desired treatment plan (15) for the at least part of the dentition (11).

3. The arrangement according to claim 1, wherein the at least one client (3) is configured to facilitate a user of the at least one client (3) to approve the treatment plan (15) or the at least one three-dimensional virtual model (10) and/or to generate a desired treatment plan (15) by adding notes (16) to the treatment plan (15) and/or
add at least one attachment (17) to the at least one three-dimensional virtual model (10) and/or
move objects (18), preferable teeth (20), of the at least one three-dimensional virtual model (10) and/or
generate interproximal reduction on the at least one three-dimensional virtual model (10) and/or
adjust the at least one three-dimensional virtual model (10) and/or
move a plurality of objects (18) of the at least one three-dimensional virtual model (10) simultaneously and/or
add cutouts or slits to the at least one three-dimensional virtual model (10).

4. The arrangement according to claim 1, wherein the at least one digital data record (14) comprises supplementary information of the at least part of the dentition (11), a virtual representation (9) of a desired orthodontic condition and/or a desired treatment plan (15), whereby it is particularly preferred that the at least one digital data record (14) is in the form of an OBJ file, an STL file, a CAD file and/or a CSV file.

5. The arrangement according to claim 1, wherein the at least one client (3) is configured to upload at least one input digital data record (19), particularly preferred an OBJ file, an STL file, a CAD file and/or a CSV file, for visualizing the at least one visual representation (9) of the at least one three-dimensional virtual model (10) and/or at least one treatment plan (15) of the at least part of the dentition (11), whereby it is particularly preferred that the at least one client (3) is configured to simultaneously upload a plurality of input digital data records (14), whereby each input digital data record (14) comprises information about a movement step of a treatment plan (15) of the at least part of the dentition (11).

6. The arrangement according to claim 5, wherein the at least one digital data record (14) and/or the at least one input digital data record (19) comprises supplementary information of the at least part of the dentition (11), preferably in the form of an intraoral scan, a scan, an x-ray, a photograph, patient data, user comments, a number of trays, comments on interproximal reductions, a number or a location of attachments (17) and/or a number of steps of the treatment plan (15).

7. The arrangement according to claim 5, wherein the at least one client (3) is configured to
identify at least one tooth (20) based on the at least one input digital data record (19) and/or the plurality of input digital data records (19) and/or
translate the at least one input digital data record (19) and/or the plurality of input digital data records (19) to the at least one virtual representation (9), preferably of movement steps of the treatment plan (15), and/or
match at least one tooth (20) with an area of the at least one three-dimensional virtual model (10), preferably of movement steps of the treatment plan (15), based on the at least one input digital data record (19) and/or the plurality of input digital data records (19).

8. The arrangement according to claim 1, wherein the at least one client (3) is configured to move and/or rotate the at least one virtual representation (9) and to modify the at least one three-dimensional virtual model (10), particularly preferred by moving at least one tooth (20) relative to at least one other tooth (20) and/or gingiva (21) and/or by locating at least one attachment (17) to at least one tooth (20).

9. The arrangement according to claim 1, wherein the at least one virtual representation (9) includes at least one attachment (17) based on a treatment plan (15), measurements of at least one tooth (20) and/or movement steps based on a treatment plan (15).

10. The arrangement according to claim 5, wherein the at least one client (3) comprises
means for importing (22), particularly preferred user interface (7), which are configured to import via a data exchange system and/or a case management system
at least one input digital data record (19) of the at least one three-dimensional virtual model (10) of the at least part of the dentition (11) to be treated and/or
at least one input digital data record (19) of a treatment plan (15) of an orthodontic treatment planning software (1) based on the at least part of the dentition (11) and/or
at least one input digital data record (14) of at least one three-dimensional virtual model (10) of at least part of the dentition (11) based on at least one digital data record of a treatment plan (15) and/or movement steps of an orthodontic treatment planning software (1) for the at least part of the dentition (11), and/or
means for processing (23) at least one input digital data record (19), particularly preferred processing unit (30) and/or interpreter between the local computer (4) or mobile device (5) and the at least one server (2), which are configured to
analyze the at least one input digital data record (19) based on the file format and/or
identify at least one object (18) of the at least part of the dentition (11) and/or at least one attachment (17) for the at least part of the dentition (11) based on the at least one input digital data record (19) and/or
match the at least one object (18) and if applicable the at least one attachment (17) to construct the at least one three-dimensional virtual model (10) of the at least part of the dentition (11) and/or
means for visualizing (24) and/or modifying, particularly preferred web browser (26) or app interface (25), which are configured
to visualize within a web browser (26) or an app (31) the at least one input digital data record (19), particularly preferred the treatment plan (15), movement steps of the treatment plan (15) and/or the at least one virtual representation (9) of the at least one three-dimensional virtual model (10) of the at least part of the dentition (11) to be treated, and/or
move and/or rotate the at least one virtual representation (9) to a desired size and/or viewing angle and/or
modify the at least one three-dimensional virtual model (10) of the at least part of the dentition (11) and/or
means for exporting (27), particularly preferred user interface (7), which are configured to export
the at least one digital data record (14) suitable for approving the treatment plan (15) or creating a new treatment plan (15) by means of the at least one server (2), whereby it is particularly preferred that the at least one digital data record (14) is in the form of a modified input digital data record (19) based on a modification of the at least one three-dimensional virtual model (10) and/or a desired modification of at least one movement step of the treatment plan (15) for the at least part of the dentition (11) and/or
a virtual representation (9) of at least one modified three-dimensional virtual model (10) and/or
the treatment plan (15), preferably with notes (16) for a desired treatment plan (15) and/or information of discrepancies and/or measurements of objects (18) of the at least one three-dimensional virtual model (10).

11. The arrangement according to claim 10, wherein
the means for importing (22) are at least temporally in signal-conducting data connection with a DDX database (28) and/or a CRM software (29), preferably by means of an API of the means for importing (22), and/or
the means for visualization (24) are configured to display discrepancies and/or measurements of objects (18) of the at least part of the dentition (11) based on the treatment plan (15) and/or on at least one modified three-dimensional virtual model (10).

12. A web browser-based or app-based method for visualizing at least one three-dimensional virtual model (10) of at least part of a dentition (11) by means of an arrangement according to claim 1, further comprising the following steps:
receiving by the local computer (4) or mobile device (5) at least one input digital data record (14) by means of the second data transmission interface (13) via a data network (32) from the first data transmission interface (12) of the at least one server (2)
visualizing by the at least one local client (3) at least one virtual representation (9) of at least one three-dimensional model (10) of the at least part of the dentition (11) by means of the user interface (7) in the form of a stand-alone web browser-based or app-based treatment plan viewer (8)
transmitting by the at least one local client (3) at least one digital data record (14) of a modified three-dimensional virtual model (10) of the at least part of the dentition (11) and/or with information for a desired treatment plan (15) of the at least part of the dentition (11) by means of the first data transmission interface (12) to the second data transmission interface (13) of the at least one server (2).

13. The method according to claim 12, wherein by means of the at least one client (3) the method
imports via a data exchange system and/or a case management system, particularly preferred by means of a user interface (7)
at least one input digital data record (19) of the at least one three-dimensional virtual model (10) of the at least part of the dentition (11) to be treated and/or
at least one input digital data record (19) of a treatment plan (15) of an orthodontic treatment planning software (1) based on the at least part of the dentition (11) and/or
at least one input digital data record (19) of at least one three-dimensional virtual model (10) of at least part of the dentition (11) based on at least one digital data record of a treatment plan (15) and/or movement steps of an orthodontic treatment planning software (1) for the at least part of the dentition (11), and/or
processes the least one input digital data record (19), particularly preferred by means of a processing unit (30) and/or an interpreter between the local computer (4) or mobile device (5) and the at least one server (3) in order to
analyze the at least one input digital data record (19) based on the file format and/or
identify at least one object (18) of the at least part of the dentition (11) and/or at least one attachment (17) for the at least part of the dentition (11) based on the at least one input digital data record (19) and/or
match the at least one object (18) and if applicable the at least one attachment (17) to construct the at least one three-dimensional virtual model (10) of the at least part of the dentition (11) and/or
visualizes, particularly preferred by means of a browser or app interface (25), within a web browser (26) or an app (31) the at least one input digital data record (19), particularly preferred the treatment plan (15), movement steps of the treatment plan (15) and/or the at least one virtual representation (9) of the at least one three-dimensional virtual model (10) of the at least part of the dentition (11) to be treated, and/or moves and/or rotates the at least one virtual representation (9) to a desired size and/or viewing angle and/or
modifies the at least one three-dimensional virtual model (10) of the at least part of the dentition (11) and/or
exports, particularly preferred by means of a user interface (7),
the at least one digital data record (14) suitable for approving the treatment plan (15) or creating a new treatment plan (15) by means of the at least one server (2), whereby it is particularly preferred that the at least one digital data record (14) is in the form of a modified input digital data record (19) based on a modification of the at least one three-dimensional virtual model (10) and/or a desired modification of at least one movement step of the treatment plan (15) for the at least part of the dentition (11) and/or
a virtual representation (9) of at least one modified three-dimensional virtual model (10) and/or
the treatment plan (15), preferably with notes (16) for a desired treatment plan (15) and/or information of discrepancies and/or measurements of objects (18) of the at least one three-dimensional virtual model (10).

14. A computer program product, which, when the program is executed by a processing unit (30), causes the processing unit (30) to carry out the method according to claim 12.

15. A non-volatile, computer-readable medium comprising instructions which, when executed by a processing unit (30), cause the processing unit (30) to carry out the method according to claim 12.
